(19) United States
(12) Patent Application Publication
(10) Pub. No.: US 2003/0012694 A1
(43) Pub. Date: Jan. 16, 2003

(54) System for the analysis of biological liquids

(76) Inventors: Bernd Roesicke, Mannheim (DE); Guenter Frey, Ellerstadt (DE)

Correspondence Address: Roche Diagnostics Corporation, 9115 Hague Road, Bldg D, P.O. Box 50457, Indianapolis, IN, 46250, US

(21) Appl. No.: 10/091,359

(22) Filed: Mar. 4, 2002

(30) Foreign Application Priority Data
Mar. 15, 2001 (DE) 101 12 507.0

(51) Int. Cl. G01N 33/00

(52) U.S. Cl. 422/58; 422/68.1; 436/164

(57) ABSTRACT

Disposable unit for single use for the analysis of biological liquids, in particular cell suspensions such as blood, urine, or sperm, with a diluent chamber (5), a sample dosage device (6), and a measuring chamber (7). The sample dosage device (6) has a dosage element (8) into which a dosage capillary (13) is integrated. The dosage element (8) is situated movably in a dosage element chamber (12) in such a manner that one opening (14) of the dosage capillary (13) is connected to a sample loading zone (17) of the disposable unit (2) when the dosage element (8) is in its first position, and when it is in its second position, the diluent chamber (5) and the measuring chamber (7) are connected to one another via the dosage capillary (13). The measuring chamber (7) has a ventilation valve (35) which is permeable to gas but impermeable to the sample liquid, so that the chamber is completely filled free of any bubbles with the liquid (36) flowing into it.

The invention relates to the analysis of biological liquids, in particular cell suspensions. The most important area of application is the acquisition of hematological data on blood. However, the invention may also advantageously be used for analyzing other cell suspensions, in particular urine or sperm. Such analyses primarily involve analyzing the sample with respect to the number (per unit volume) and morphology of the cells dispersed in the cell suspension. In principle, however, the invention may also be used for other analytical tests of biological liquids. Hereafter, without limiting its general usefulness, reference is made to the field of application in hematology.

Hematological data of blood samples are traditionally obtained visually by means of counting cells in a cell chamber. This method requires very well-trained laboratory personnel. Even if this requirement is satisfied, it is tedious, slow, and susceptible to errors.

For the automated analysis of blood samples flow through counters are used in which the sample liquid flows through a narrow channel, generally after preliminary dilution, and the passage of the cells is detected by means of an impedance detector arranged on the channel. Cell counts can be derived from the number of pulses that are generated by the detector when the cells pass the measuring point and from the volumetric flow rate. The form of the pulses can be processed into information about the morphology of the cells. Recently, flow through counters having an optical detector instead of an impedance detector have been developed. In this optical detector, detection is based on the deflection of a laser beam by the cells.

Since the investment costs for such devices are high, as is the required qualification of the operators, this method is not well-suited for on-site use, in particular at the doctor's office or decentralized at hospital stations.

Based on this, the problem addressed by the invention is to simplify analytical testing, in particular of biological cell suspensions, so that on the one hand the required investment costs are significantly reduced, and on the other hand a good level of accuracy of the analytical results is achieved with a simple operation.

The object is achieved by a disposable unit for one-time use for the analysis of biological liquids, in particular cell suspensions such as blood, urine, or sperm, containing a diluent chamber, a sample dosage device, and a measuring chamber, wherein the sample dosage device has a dosage element into which a dosage capillary running between two openings is integrated. The dosage element is arranged within a dosage element chamber formed in the disposable unit in such a manner that, when the dosage element is in its first position, one opening of the dosage capillary is connected to a sample loading zone of the disposable unit, and in its second position, one of the openings of the dosage capillary is connected to the diluent chamber and the other opening of the dosage capillary is connected to the measuring chamber. Thereby, in the second position, the diluent chamber and the measuring chamber are connected to one another via the dosage capillary. The measuring chamber has a defined volume and is provided with a ventilation valve which is permeable to gas but impermeable to the sample and diluent liquids, so that the chamber is completely filled free of any bubbles by the liquid flowing into it.

The invention is also directed to a system comprising disposable units of the type described above and an analysis instrument with a mounting unit to position a disposable unit in its measuring position; an actuator that operates on the diluent chamber in such a manner that the liquid diluent contained therein is placed under pressure and consequently flows through the dosage capillary into the measuring chamber when the dosage element is in its second position; a device to detect a physical property of the liquid contained in the measuring chamber of the disposable unit positioned in its measuring position; and an evaluation device for deriving test results based on the result of the detection of the physical property.

Furthermore, the invention relates to a method of analyzing biological liquids by means of a disposable unit of the type described above, wherein the sample liquid is placed in contact with the sample loading zone of the disposable unit in such a manner that it is sucked into the dosage capillary by capillary forces when the dosage element is located in its first position. Thereafter the dosage element is moved to its second position and pressure is exerted on the liquid diluent in the diluent chamber in such a manner that it flows into the measuring chamber via the dosage capillary, whereby the sample liquid is flushed out of the dosage capillary into the measuring chamber. The measuring chamber is filled completely and free of bubbles by the sample liquid flushed out of the dosage capillary and by the diluent liquid while the gas displaced by the incoming liquids escapes through the ventilation valve which is permeable to gas, but impermeable to the sample and diluent liquids. A physical property of the liquid, which is thereafter contained in the measuring chamber (“test liquid”), is measured and analyzed to derive the test result.

The term “disposable unit” (also simply “disposable”) is used in medical technology to designate items and devices intended for one-time use. In the case of the present invention, the disposable unit comprises few parts and may be manufactured cost-effectively from plastics, in particular by means of an injection molding method.

In spite of the low manufacturing cost the dilution ratio is very precise and reproducible. It depends only on the volumes of the dosage capillary and of the measuring chamber. Those parts which define these volumes can be manufactured with standard plastics injection molding techniques with an exactly defined chamber volume.

Dosing by means of a measuring chamber which is located downstream from the dosage capillary is particularly advantageous if the disposable unit contains the diluent liquid pre-packaged by the manufacturer. Even if the plastics material used is highly gas tight, a loss of pre-packaged diluent liquid from the diluent chamber has to be expected during the long storage time (often more than two years) which is customary in laboratory technology. With a device according to the invention such loss of liquid can easily be taken into account by making the volume of the diluent inside the diluent chamber correspondingly larger than the volume of the measuring chamber. Thus the precision of the analysis is not reduced by losses of diluent liquid during storage.

Practical tests have shown that (with an embodiment in which the measuring chamber is equipped with an observation window to perform optical analysis) the information required in hematology about the number and morphology of cells contained in blood can be obtained in a simple manner. The high level of accuracy achieved in these experiments shows that despite the simple design a quick, homogenous distribution of the entire volume of blood contained in the dosage capillary in the liquid diluent can be achieved.

To the extent the invention is used for the examination of cell suspensions, the test results are based on microscopic observation of the liquid contained in the measuring chamber after the dilution process. This observation is preferably conducted electronically by means of an image converter chip integrated into a microscope used for detection and an associated evaluation electronics. Suitable techniques have been developed for automatically monitoring the growth of biochemical cell cultures and allow detection and evaluation of the relatively fast moving cells in the diluted cell suspension. They are described inter alia in the following publications:

1) DE 4032002 C2

2) H. Suhr et al. “In Situ Microscopy for On-Line Characterization of Cell-Populations in Bioreactors, Including Cell-Concentration Measurements by Depth from Focus”, Biotechnology and Bioengineering, 1995, 106 to 116

3) DE 19726518 A1

4) EP 0990936 A1

5) DE 19923074 A1

The method described in these publications as “in situ microscopy (ISM)” is also suitable for the present invention. In this respect, their contents are incorporated by reference into the present application.

The invention is described in greater detail with reference to exemplary embodiments shown in the figures. The features shown and described may be used individually or in combination with one another to create preferred embodiments of the invention.

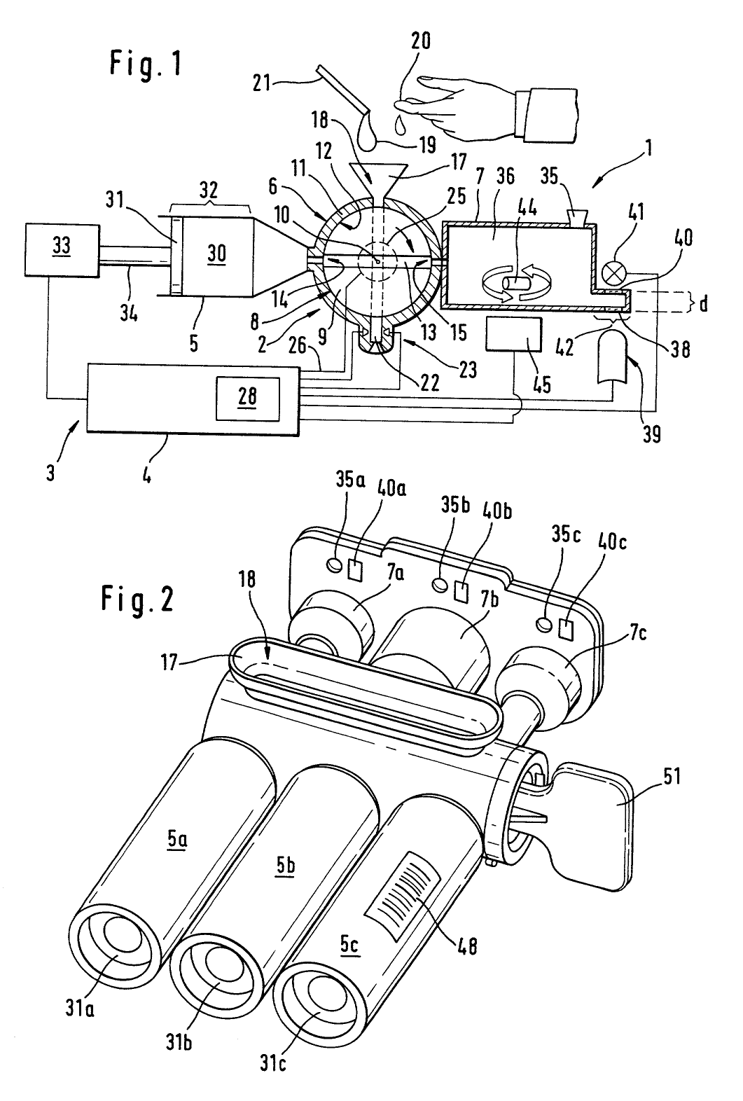

FIG. 1 shows a highly schematized diagrammatic drawing detailing the invention,

FIG. 2 shows a perspective drawing of a disposable unit for explanation of the invention,

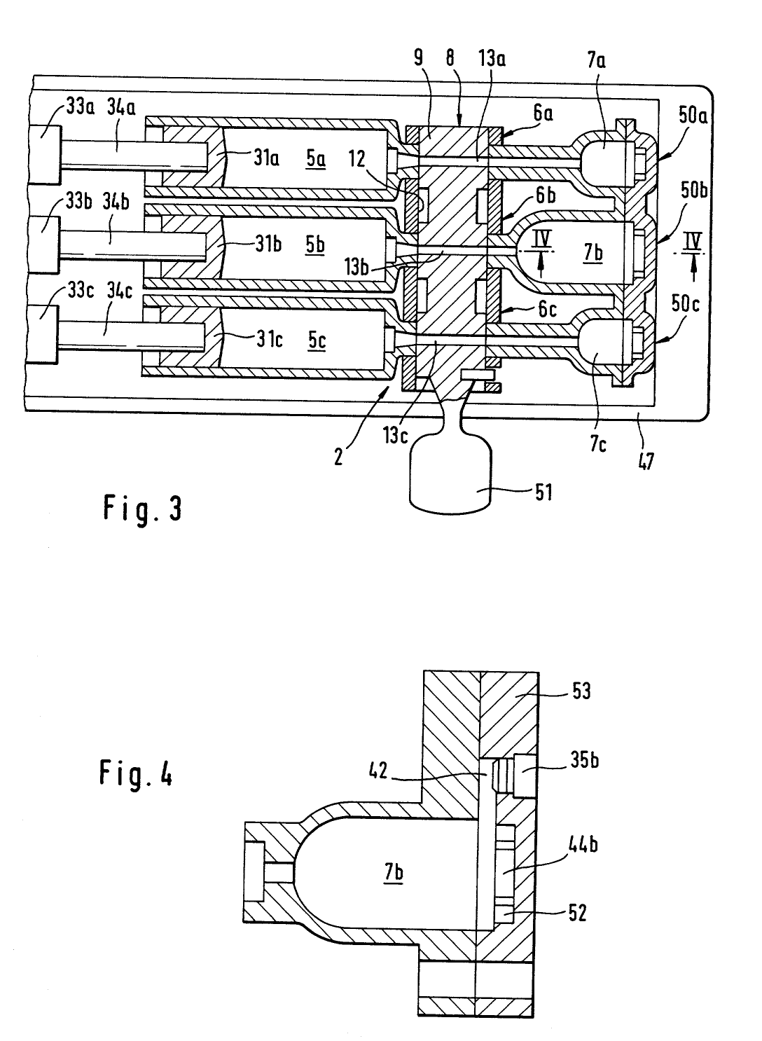

FIG. 3 shows a longitudinal section of a disposable unit according to FIG. 2,

FIG. 4 shows a sectional cut through a measuring chamber along the line IV-IV in FIG. 3.

FIG. 1 shows symbolically a system for analyzing cell suspensions with respect to the number and morphology of the cells contained therein. It is in particular suitable for determining hematological parameters (for example, RBC, HCT, MCV, HGB, MPV and WBC) in blood samples. It is designated hereafter as a hematology system 1 and essentially comprises a disposable unit 2, which functions to dilute and otherwise prepare the sample, and an analysis instrument 3 for performing optical measurements and for electronically deriving the test results therefrom. The analysis instrument contains measurement and evaluation electronics 4 and various mechanisms to mount and activate a disposable unit 2, which mechanism will be described in greater detail below. For the sake of clarity, the housing of the analysis instrument 3 is not shown.

Components of the disposable unit 2 include a diluent chamber 5, a sample dosage device 6, and a measuring chamber 7. The sample dosage device 6 has a sample dosage element 8, which in the embodiment shown is formed as a rotor element 9 and is arranged and adapted to be rotated around an axis 10 inside a dosage element chamber 12 formed by a rotor housing 11. A dosage capillary 13 with two openings 14 and 15 is integrated into the rotor element 9. The rotor element 9 can be adjusted between (at least) two positions, which differ with respect to the orientation of the dosage capillary 13. In the first position one opening 14 of the dosage capillary 13 is connected to the sample loading zone 17 in such a manner that a blood sample present therein flows, driven by capillary forces, into the dosage capillary 13 and fills it completely. The position of the dosage capillary 13 in this first position of the rotor element 9, which is hereafter called the “filling position”, is shown with dashed lines in FIG. 1.

The sample loading zone 17 can be embodied in various ways. It is only essential that it be suitable for applying the sample in such a manner that it flows into the dosage capillary 13 when the rotor element is in its filling position. An embodiment in the form of a funnel-shaped sample intake chamber 18 is convenient, the chamber being formed in such a manner that it may easily be filled (for example, directly by a grooved finger 20 or by means of the tip of a pipette 21) with the required volume of sample liquid 19.

Since complete filling of the dosage capillary 13 is extremely important for the accuracy of the analytical results, a control capillary 22 with an electronic filling control device 23 (for example, in the form of a photoelectric barrier) is arranged in the rotor housing 11 in a position aligned with the second opening 15 when the rotor element 9 is in its filling position. The filling control device 23 indicates when the blood sample flows into the control capillary 22 and consequently when the dosage capillary 13 has been completely filled.

In the second position (“flow position”) of the rotor element 9, the dosage capillary 13 is located in the position shown with full lines, in which it is connected on one side to the diluent chamber 5 (via opening 14) and on the other side to the measuring chamber 7 (via opening 15), so that the diluent chamber 5 and the measuring chamber 7 are connected to one another via a continuous liquid channel.

The dosage capillary 13 is filled by a precisely specified volume of sample liquid, which is flushed into the measuring chamber 7 and used for the desired analysis. This principle of dosing a defined volume is known from various applications in laboratories. For example in DE 3507032 C2, a device for the volumetric measurement and transfer of a sample from one chamber into another chamber is described. In that device, the dosage capillary is integrated into a slidable transfer element, which in two different embodiments can either be moved transversely to the direction of the dosage capillary or pivoted between different rotational positions in a corresponding snug fit. As with this known device, the dosage element of the invention can also be a component making a translational sliding movement inside a suitable housing unit. However, the embodiment as a rotor element shown in the figures is preferred.

In a hematological liquid flow through counter with impedance detection which is sold by the company SWELAB Instrument AB, Sweden, under the name “AutoCounter” a so-called “fluid distribution valve” that also has an integrated dosage capillary is used. This fluid distribution valve is a high-precision, stainless steel component and functions to inject a precisely reproducible sample volume of 20 μl into the fluid stream.

Such a device is also described in U.S. Pat. No. 6,284,548. The diluent liquid is dosed by means of a high precision piston-cylinder unit located upstream from the dosage capillary and is pressed into the dosage capillary. Such a design is not suitable for disposable units which have to be manufactured at low cost.

The diluent chamber 5 contains a liquid diluent 30, which is preferably pre-packaged by the manufacturer (thus, already prepared for its desired purpose and filled into the diluent chamber 5 by the manufacturer of the disposable unit), whereby the disposable unit 2 is ready for use without any further preparations. As noted above, the precision of the dilution (and thus the precision of the resulting analytical results) is independent of any losses of diluent liquid from the diluent chamber which may occur during storage.

The diluent chamber 5 is designed in such a manner that the liquid diluent 30 located therein can be placed under pressure so that it flows, when the rotor element 9 is in its flow position, into the measuring chamber 7 via the dosage capillary 13. To that end, the diluent chamber 5 has a movable piston 31, which can be moved axially inside a corresponding cylindrically shaped housing section 32 of the diluent chamber 5. A linear drive 33 functions as an actuator to move the piston 31. It is a component of the analysis instrument 3 controlled by its measurement and evaluation electronics 4 and operates on the piston 31 via a cylinder rod 34. In the context of the invention, however, other pressure-generating mechanisms for the disposable unit can also be used. In particular, it may be favorable to form the walls of the diluent chamber 5 from an easily deformable material (for example, a plastic sheet) so that a pressure in the liquid diluent 30 can be generated by exterior pressure onto the walls. The accuracy of the dosed amount is independent of the volume of the diluent chamber 5 and therefore is not affected by this cost-effective embodiment.

When the liquid diluent 30 flows through the dosage capillary 13, it completely flushes the sample liquid contained therein into the measuring chamber 7. The measuring chamber 7 has a ventilation valve 35 which is permeable to gas but impermeable to the sample liquid and is arranged, when the disposable unit is in operating position, at the uppermost part of the measuring chamber 7. Suitable ventilation valves are offered for sale and are also called “hydrophobic vents” in the language of the art. The ventilation valve 35 ensures that the measuring chamber is completely filled free of any bubbles, so the volume of liquid contained in the measuring chamber 7 at the end of the filling process is precisely determined by the volume of the chamber. The mixture ratio of sample liquid 19 and liquid diluent 30 is consequently defined by the volume of the dosage capillary 13 and by the volume of the measuring chamber 7. The resulting mixture in the measuring chamber 7 is hereafter called test liquid 36.

In order to enable optical analysis of the test liquid 36, the measuring chamber 7 has an observation window 38, onto which a light-optical detection device 39 is focused. A light source 41 is provided for illumination. In the embodiment shown, this light source radiates into the test liquid 36 through a separate-illumination window 40.

According to a preferred embodiment, the observation window 38 is located in a measuring area 42 of the measuring chamber 7 having a smaller liquid layer thickness (when measured perpendicular to the surface of the observation window 38) than the rest of the measuring chamber. It is preferable for the thickness d of the liquid layer in the measuring area to be no more than 1 mm, 0.5 mm being particularly preferable. Preferably the volume of the liquid in the measuring area is at most one third of the total volume of the liquid in the measuring chamber 7, one tenth being particularly preferable.

The optical detection device 39 is preferably an electronic microscope to observe cells contained in the test liquid. In situ microscopy (ISM), known from citations 1 to 5, is particularly suitable.

In the context of the invention, it was determined that the ISM method is, in conjunction with the system shown, very well-suited for determining hematological parameters. In contrast to the previously known cell chamber counting method, the analysis is performed automatically. No specially trained personnel are required. In contrast to previously known flow through counters, the cost of the equipment required for the analysis instrument 3 is very low, whereby such a system can economically be used decentralized at hospital stations or doctors'offices.

It is highly important for the accuracy of such a method that the test liquid 36 be mixed so well throughout the entire measuring chamber 7, including the measuring area 42, that the content of the measuring area 42 is statistically representative of the overall content of the measuring chamber 7. To this end, an agitator is provided for stirring the test liquid 36 contained in the measuring chamber 7. In the case shown, a magnetic agitator 44 which is caused to rotate by a solenoid actuator 45 is contained in the measuring chamber 7. The actuator is a component of the analysis instrument 3 and is controlled by the measurement and evaluation electronics 4. Alternatively, other contact-free mixing principles may be used which are suitable for causing through the closed wall of the measuring chamber 7 a mixing movement of the test liquid 36, in particular an ultrasonic mixer.

When a blood sample 19 is to be analyzed with the system 1 shown, it is inserted into the funnel-shaped sample intake chamber 18 of the sample loading zone 17, and it flows into the dosage capillary 13, the rotor element 9 being in its filling position. When the dosage capillary 13 is completely filled, the filling control device 23 generates a signal which is transmitted to the analysis instrument 3. Then, the rotor element 9 is turned to its second position. To this end preferably an electrical rotor actuator 25 is used, which is shown with dashed lines in FIG. 1 and is controlled via a wire 26 from the analysis instrument 3. Alternatively, it is also possible to turn the rotor element 9 manually. In this case the operator can be prompted to turn the rotor element 9 by a corresponding display 28 on the analysis instrument 3.

When the rotor element 9 is in its filling position, the linear drive 33 is set in motion and forces liquid diluent 30 into the measuring chamber 7 via the dosage capillary 13, the displaced air escaping through the ventilation valve 35. This process is continued until the measuring chamber 7 has been completely filled. At this point, the pressure in the system and thus in the diluent chamber 5 increases sharply. This pressure increase can be detected by known methods and used to switch off the linear drive 33.

Finally, the agitator 44,45 is activated in order to thoroughly mix the test liquid 36 in the measuring chamber 7 and take a measurement thereof, preferably an ISM reading. The signals from the light-optical detection device 39 are transmitted to the measurement and evaluation electronics. The result of the electronic evaluation is indicated as test result on the display 28 via data transfer or some other method.

FIGS. 2 to 4 are accurate scale drawings of a disposable unit 2 that was used for the experimental testing of the invention. Elements whose function corresponds to those of FIG. 1 are designated by the same reference numbers. Additionally, a mounting unit 47 is shown in FIG. 3, which is a component of an analysis instrument 3 and in which a disposable unit is fixed in its measuring position.

An additional feature of the disposable unit shown in FIGS. 2 to 4 is the fact that it has a plurality of measuring channels 50a to 50c, each of which contains a diluent chamber 5a to 5c, a sample dosage device 6a to 6c, and a measuring chamber 7a to 7c. The dosage capillaries 13a to 13c are integrated into a common dosage element 8, which is formed as a rotor element 9 like that in FIG. 1. The rotor element is mounted rotatably in a rotor chamber 12. The remaining multiple components of the measuring channels 50a to 50c are designated by the additional letters a to c. The rotor element 9 is turned between its filling position (not shown) and its flow position (shown) by means of a turning handle 51.

Because a plurality of measuring channels 50a to 50c is integrated into a common disposable unit 2, it is possible to perform in parallel different reaction sequences based on one sample, wherein the diluent chambers 5a to 5c generally do not only contain an inert liquid (in particular water), but also reactive ingredients. In the context of hematology, for example, it may be useful if one of the liquid diluents in the diluent chambers contains a lytic reagent by means of which red blood cells are dissolved. It may also be useful if the measuring chambers 7a to 7c differ with respect to their volumes in order to achieve different levels of dilution. Preferably a machine-readable code 48 containing important information required for the evaluation of the test results is arranged on the disposable unit, in particular for the identification of the contents of the reagent liquid chambers.

As may be seen from FIGS. 2 and 3, the rotor element 9 has a cylindrical outer surface and is situated in a corresponding cylindrically shaped rotor chamber 12. Regardless of the form of the dosage element 8 chosen in each instance, its surface, at least the area around the openings 14, 15 of the dosage capillary 13, should slide in contact with the surrounding surface of the dosage element chamber which houses the dosage element. Thus the dosage capillary is closed during movement from the filling position to the flow position, whereby the volume of fluid contained therein remains constant during this movement.

Additional details of a preferred embodiment of the measuring chamber may be seen in FIG. 4. The magnetic agitator 44b is preferably situated in a corresponding recess 52 of the inner space of the measuring chamber. In the measuring position shown, the ventilation valve 35b is arranged on the uppermost part of the measuring chamber 7b, which in the embodiment shown at the same time forms the measuring area 42. The observation window, which is also arranged in this measuring area 42, is positioned in the plane of the cover element 53 in the vicinity of the ventilation valve 35b, but it cannot be seen in FIG. 4, because it does not lie on the sectional plane shown therein.

1. Disposable unit for single use for the analysis of biological liquids, in particular cell suspensions such as blood, urine, or sperm, comprising a diluent chamber, a sample dosage device, and a measuring chamber, wherein
the sample dosage device has a dosage element into which a dosage capillary running between two openings is integrated,
the dosage element is arranged inside a dosage element chamber formed in the disposable unit and is movable in such a manner that one opening of the dosage capillary is connected to a sample loading zone of the disposable unit when the dosage element is in a first position, and that, when it is in a second position, one of the openings of the dosage capillary is connected to the diluent chamber and the other opening of the dosage capillary is connected to the measuring chamber, so that the diluent chamber and the measuring chamber are in the second position connected to one another via the dosage capillary, and
the measuring chamber has a defined volume and is provided with a ventilation valve which is permeable to gas but impermeable to the sample and diluent liquids, so that the measuring chamber is completely filled free of bubbles by the liquid flowing into it.

2. Disposable unit according to claim 1, comprising a plurality of measuring channels, each comprising a diluent chamber, a sample dosage device, and a measuring chamber.

3. Disposable unit according to claim 2, wherein a plurality of dispensing capillaries of the sample dosage device are integrated into a common dosage element.

4. Disposable unit according to claim 1, in which the dosage element is formed as a rotor element mounted rotatably in the dosage element chamber.

5. Disposable unit according to claim 1, wherein the diluent chamber is filled with a liquid diluent pre-packaged by the manufacturer of the disposable unit and the volume of the pre-packaged diluent is larger than the volume of the measuring chamber.

6. Disposable unit according to claim 1, wherein the measuring chamber contains an agitator with contact-free activation, in particular a magnetic agitator.

7. Disposable unit according to claim 1, wherein the measuring chamber has an observation window to allow optical analysis of a liquid contained in the measuring chamber.

8. Disposable unit according to claim 7, wherein the measuring chamber includes a measuring area with a liquid layer formed by a partial volume of the measuring chamber, the liquid layer having, in comparison to the rest of the measuring chamber, a small volume and a small thickness measured perpendicular to the surface of the observation window.

9. Disposable unit according to claim 8, wherein the volume of the liquid layer in the measuring area is no more than one third, preferably no more than one tenth, of the volume of the measuring chamber.

10. Disposable unit according to claim 8, wherein the thickness of the liquid layer in the measuring area is no more than 1 mm, preferably no more than 0.5 mm.

11. Disposable unit according to claim 1, comprising a machine-readable code that contains information pertinent to the analysis of the test results.

12. System for the analysis of biological liquids, in particular cell suspensions such as blood, urine, or sperm, comprising disposable units according to any one of the preceding claims and an analysis instrument, the analysis instrument comprising
a mounting unit for positioning a disposable unit in a measuring position,
an actuator, which operates on the diluent chamber in such a manner that the liquid diluent contained therein is placed under pressure and consequently flows into the measuring chamber via the dosage capillary when the dosage element is in its second position,
a device for detecting a physical property of a liquid contained in the measuring chamber of the disposable unit positioned in its measuring position,
the measuring chamber is filled completely and free of bubbles by the sample liquid flushed out of the dosage capillary and by the diluent liquid while the gas displaced by the incoming liquids escapes through the ventilation valve which is permeable to gas but impermeable to the sample and diluent liquids, and
an evaluation device for deriving test results based on the result of the detection of the physical property.

13. System according to claim 12, wherein the analysis instrument has an actuator for moving the dosage element between its first position and its second position.

14. System according to claim 12, wherein the analysis instrument has an agitator for mixing the liquid contained in the measuring chamber.

15. System according to claim 12, wherein the device for detecting a physical property comprises an optical detection device, in particular a microscope for the microscopic analysis of liquid contained in the measuring chamber.

16. Method for the analysis of biological liquids, in particular cell suspension such as blood, urine, or sperm, by means of a disposable unit according to any of one claims 1 to 11, wherein
the sample liquid is placed in contact with the sample loading zone of the disposable unit in such a manner that it is sucked into the dosage capillary by capillary forces when the dosage element is located in its first position,
the dosage element is moved to its second position,
pressure is exerted on liquid diluent contained in the diluent chamber in such a manner that it flows into the measuring chamber via the dosage capillary, whereby the sample liquid is flushed out of the dosage capillary into the measuring chamber, and
a physical property of the liquid, which is thereafter contained in the measuring chamber, is measured and analyzed to derive the test result.

17. Method according to claim 16, wherein the measurement of the physical property comprises microscopic analysis of the liquid contained in the measuring chamber.

18. Method according to claim 17, wherein the microscopic analysis is performed by means of an electronic camera, and the image from the electronic camera is electronically analyzed with respect to the number and morphology of cells contained in the measuring chamber.
